(19) United States
(12) Patent Application Publication
(10) Pub. No.: US 2026/0102270 A1
(43) Pub. Date: Apr. 16, 2026

(54) Orthopedic Device with Mechanical Advantage Closure

(71) Applicant: Ricker Angus Miles, Anchorage, AK (US)

(72) Inventor: Ricker Angus Miles, Anchorage, AK (US)

(21) Appl. No.: 18/911,704

(22) Filed: Oct. 10, 2024

(51) Int. Cl. A61F 5/01

(52) U.S. Cl. CPC A61F 5/0123; A61F 2005/0132

(57) ABSTRACT

An orthopedic device having a mechanical advantage closure in the form of an orthopedic primary strap device capable of attaining a theoretical 3:1 mechanical advantage when cinching and securing the primary strap device. The actual mechanical advantage attained is approximately 2:1 because frictional loss occurs between fabric components while tightening the device. The orthopedic primary strap device can be utilized as a unitary brace, or one or more primary strap devise can be incorporated into an orthopedic sleeve to provide about a 2:1 mechanical advantage in compression closure.

### FIELD OF INVENTION

The present invention relates to orthopedic devices and particularly to braces and supports having a mechanical advantage closure compression support system.

### BACKGROUND OF THE INVENTION

There are many products on the market that are designed to provide support to knees, ankles, quadriceps, elbows, wrists, and other injury-prone areas of the human body. These devices typically are designed to help prevent or alleviate a variety of joint and muscle maladies, including for example pertaining to the knee joint, patellar tendonitis, and Osgood Schlatter's disease. Reusable products of this kind typically are composed of a material that stretches, such as the conventional ace bandage or other stretchable materials that are tightened with a conventional Velcro™ fastening means. Other products are in the form of compression braces composed of neoprene that are designed for a particular joint, such as an ankle, elbow, wrist, or knee.

However, the closure design for these types of products do not provide any actual mechanical advantage greater than 1:1. As a result, many athletes wrap their ankles with cloth athletic tape to prevent injury and provide support during football, tennis, basketball, soccer, running, and other sports. Cloth athletic tape does not stretch, can be tightly wrapped, has an adhesive side, and does not slip or stretch. Similarly, some professional athletes with knee issues use athletic tape below the patella, wrapping the tape tightly so it will not slip or stretch. While the use of athletic tape provides an effective support, it is a one-time use product and cannot be re-used. Athletic tape also leaves an adhesive residue on the skin when it is removed, along with tearing away hair and can create a skin rash. Plus, athletic tape is not comfortable and can be quite expensive for daily use.

### PRIOR ART

The prior art is replete with braces and stabilizers for joints, particularly knee joints, as exemplified by U.S. Pat. No. 4,940,044 to Castillo, U.S. Pat. No. 6,066,100 to Nauert, U.S. Pat. No. 6,413,232 B1 to Townsend et al., U.S. Patent Application Publication 2019/0358072 A1to Larouche, U.S. Patent Application Publication 2021/0205111 A1 to Johnson et al. and WO Patent Publication 2022/046043 A1 to Johnson et al.

Braces have been developed which recognize the need for a selective force to exert pressure. For instance, U.S. Patent Application Publication 2017/0252200 A1 to Taylor provides a knee brace which includes at least one force device in the form of an attachment strap. U.S. Pat. No. 11,529,251 B2 to Garth et al. provides a brace for the lower spine having both a lumbar support and a tightening system in the form of pulled cords. And, U.S. Pat. No. 7,819,803 B2 to Sindel at al. relates to a mechanical advantage closure system for a knee brace comprising a closure unit with a pull system to provide a mechanical advantage compression force to the knee.

Despite the many advances and treatments of the prior art, a need still exists for a device to provide compression support to a joint or other injury prone area of the human body which is capable of quickly and easily applying a theoretical 3:1 mechanical advantage. Actual mechanical advantage is approximately 2:1 because frictional loss occurs between fabric components while tightening the device. Such a support device should be characterized by minimal stretch and should enable the user to cinch the support means to the desired level of compression and comfort with minimal effort. Further, such a device should be reusable. And, such a support device should be capable of being used as a unitary primary strap device or in combination with an orthopedic sleeve.

### SUMMARY OF THE INVENTION

The invention of the present subject matter relates to an orthopedic device in the form of a primary strap device which provides about a 2:1 mechanical advantage of closure compression support to the user.

Accordingly, it is an object of the present subject matter to provide an orthopedic device with a mechanical advantage closure which can be used as an orthopedic primary strap device, such as a knee strap, which can be pulled and cinched to attain an approximate 2:1 mechanical advantage.

It is another object of the present subject matter to provide an orthopedic device with a mechanical advantage closure which is characterized by minimal stretch, can be tightly wrapped, and does not slip.

It is still another object of the present subject matter to provide an orthopedic device with a mechanical advantage closure which can be quickly and easily used to achieve about a 2:1 mechanical advantage closure compression with minimal effort.

It is a yet another object of the present subject matter to provide an orthopedic device with a mechanical advantage closure which permits the user to attain the desired level of compression and comfort.

It is a further object of the present subject matter to an orthopedic device with a mechanical advantage closure in the form of a primary strap device which can be used to provide compression support below a knee or below an elbow.

It is yet a further object of the present subject matter to provide an orthopedic device with a mechanical advantage closure which can be used in combination with an orthopedic sleeve.

It is still a further object of the present subject matter to provide an orthopedic device with a mechanical advantage closure which uses hook and loop fasteners.

It is an additional object of the present subject matter to provide an orthopedic device with a mechanical advantage closure which is composed of a rip-stop, low stretch material fabric.

It is an additional object of the present invention to provide a mechanical advantage closure compression support system which can be used to provide orthopedic support to knees, ankles, quadriceps, elbows, wrists, and other injury-prone areas of the human body.

These and other objects of the present subject matter are accomplished by providing an orthopedic device with a mechanical advantage closure in the form of a primary strap device comprising an elongated strap member having a proximal end, a mid-section along its elongated length, and a distal end, an arm member which is attached to and extends from the mid-section of the strap member, the arm member having a tip end, the proximal end strap member and the tip end of the arm member further comprising slotted openings which may be reinforced with rectangular metal rings sewn therein that are configured to fit through each other by turning one of the rings sideways. The distal end of the strap member is provided with a plurality of micro hook fasteners, corresponding to a plurality of micro loop fasteners located on the same side of the strap member at the mid-section. In operation, the distal end of the strap member having the micro hook fasteners is introduced and pulled through the slotted openings, and cinched to attain about a 2:1 mechanical advantage.

### BRIEF DESCRIPTION OF DRAWINGS

FIG. 1 is a top perspective view of the mechanical advantage closure compression support system of the present subject matter.

FIG. 2 is a top perspective view of the mechanical advantage closure compression support system of the present subject matter depicting the method of creating the mechanical advantage with the strap member.

FIG. 3 is a side perspective view of the mechanical advantage closure compression support system of the present subject matter depicting the resulting creation of the mechanical advantage with the strap member.

FIG. 4 is side perspective view of the orthopedic device with a mechanical advantage closure of the present subject matter in the form of a compression strap wrapped about a user's leg immediately under the patella.

FIG. 5 is a side perspective view of the orthopedic device with a mechanical advantage closure of the present subject matter utilizing two strap members attached to a sleeve and wrapped about the quadriceps femoris of a user.

FIG. 6 is side perspective view of the orthopedic device with a mechanical advantage closure of the present subject matter utilizing three strap members attached to a sleeve wrapped about the ankle of a user.

### DETAILED DESCRIPTION OF THE INVENTION

In the following description, like reference numbers are used to identify like elements. Furthermore, the drawings are intended to illustrate major features of exemplary embodiments in a diagrammatic manner. The drawings are not intended to depict every feature of every implementation nor relative dimensions of the depicted elements and are not drawn to scale. In the following description, numerous specific details are set forth to clearly describe various specific embodiments disclosed herein. One skilled in the art, however, will understand that the presently claimed invention may be practiced without all the specific details discussed below. In other instances, well known features have not been described so as not to obscure the invention.

The terms “compression support device”, “compression support brace”, “compression support system”, “strap support system”, “mechanical advantage closure compression support system”, “MACCSS”, derivative terms thereof, and similar terms may be used throughout the specification and it is to be understood that such terms are interchangeable and all refer to the orthopedic device with mechanical advantage closure of the present subject matter.

Also, it is to be understood that the phraseology and terminology used herein is for the purpose of description and should not be regarded as limiting. The use of the terms “including”, “comprising”, “having”, and variations thereof herein is meant to encompass the items listed thereafter and equivalents thereof as well as additional items.

The mechanical advantage closure compression support system of the present subject matter is inspired by a trucker's hitch which enables a strap member to be tightened to achieve additional mechanical advantage. Indeed, the utilization of the trucker's hitch provides about a 2:1 mechanical advantage. The closure compression support system also is inspired by the dynamic power strap on a ski boot which enables super tight cinching of the elastic power strap.

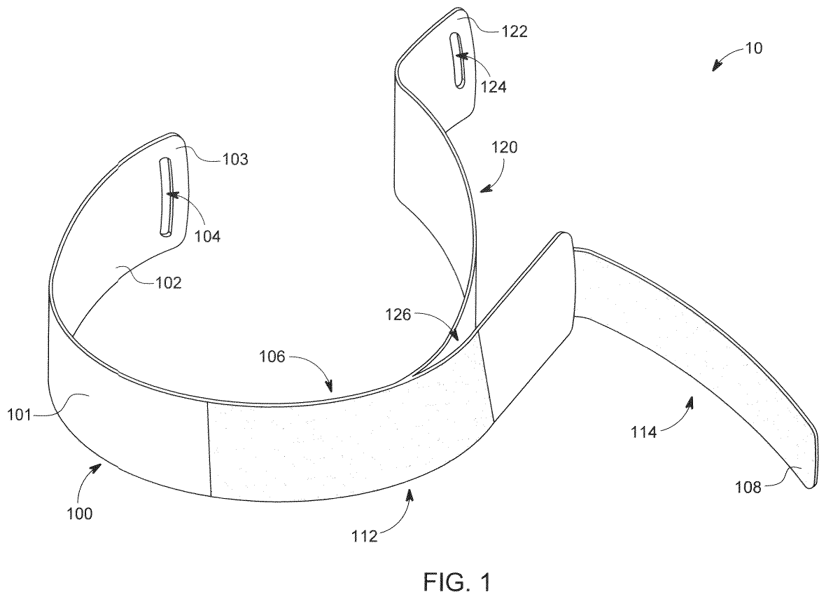

Accordingly, the orthopedic device with mechanical advantage closure of the present subject matter comprises a primary strap device. Referring to FIG. 1, the orthopedic device with mechanical advantage closure 10 is in the form of a strap member 100 having an elongated length with an upper side 101, an underside 102, a proximal end 103 provided with a slotted opening 104, a mid-section 106 having a plurality of micro loops 112 of a hook and loop fastening means disposed on the upper side 101, a distal end 108 having a plurality of micro hoops 114 of a hook and loop fastening means disposed on the upper side 101, and an arm member 120 attached to the underside 102 of the mid-section and extending outwardly therefrom, the arm member having a tip end 122 provided with a slotted opening 124 distal from the mid-section. The arm member 120 is sewn to the strap member 100 by stitching 126. Preferably, opening 104 is reinforced by a ring element, such as a rectangular metal or durable plastic D-ring. Similarly, opening 116 is reinforced with a ring element, such as a rectangular metal or durable plastic D-ring. Both ring elements are sewn or otherwise secured within the slotted openings 103 and 124. In a preferred embodiment, tip end 122 is sheathed in a plastic material, the slotted opening being reinforced with the D-ring. In an alternate embodiment, each of the openings may be in the form of a small section of reinforced, rigid fabric with a rectangular opening.

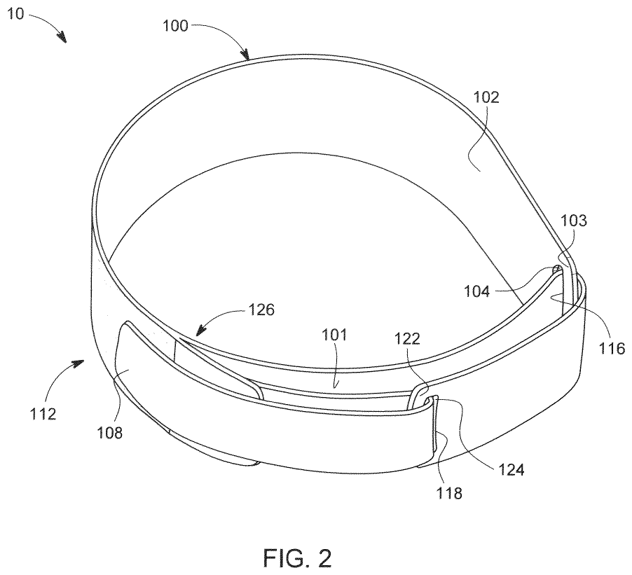

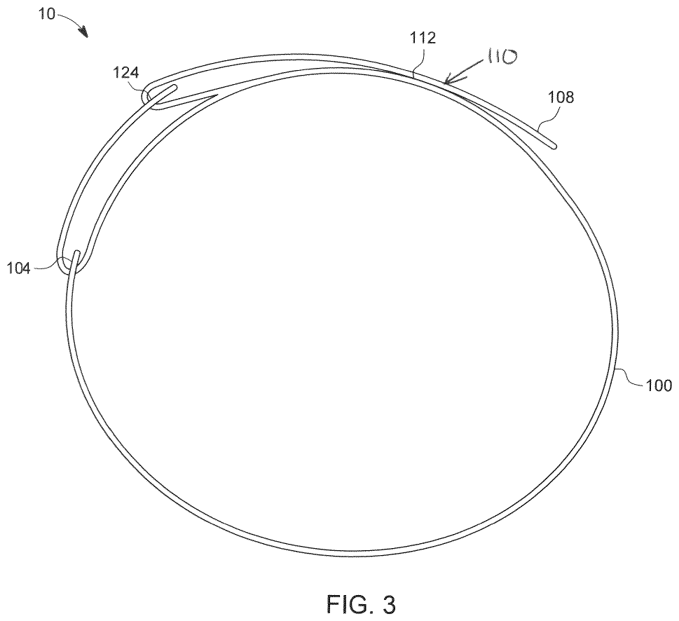

FIG. 2 illustrates the operation of the primary strap device to attain the 2:1 mechanical advantage of the compression closure. In the first step, the distal tip end 122 is turned sideways, introduced into the slotted opening 104 of the proximal end 103 and fed through the opening 104. In the second step, the distal end 108 is introduced into the slotted opening 124 of the tip end 122 and is continually pulled through the opening 124 such that it folds back onto itself in such a manner that the plurality of micro hoops 114 engage and are detachably secured to the plurality of micro loops 112 as the strap member is pulled and cinched with about a 2:1 mechanical advantage to attain a desired level of compression. The MACCSS is cinched and detachably secured by the hook and loop fastening means 110 as illustrated in FIG. 3.

The strap member may be composed of any material that is characterized as having minimal stretch. Preferably, the strap member is composed of a ripstop-type fabric which is lightweight, tear resistant, water resistant, and extremely durable material. Suitable ripstop materials may be composed from polyester, nylon or natural fibers and are designed to resist tearing and wear with reinforcing threads woven in a checkered pattern. However, it will be obvious to those skilled in the art that the strap member can be composed of any suitable material that has minimal stretch, is tear resistant and can provide sufficient tensile strength to apply compressive force to the underlying tissue.

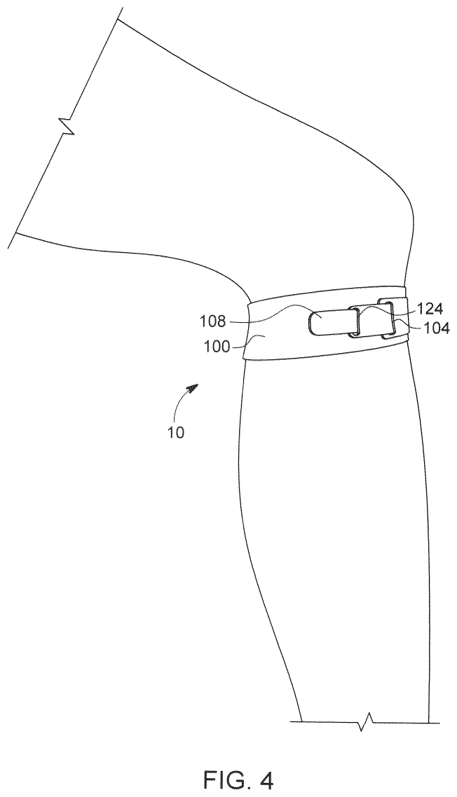

The orthopedic device with a mechanical advantage closure of the present subject matter can be utilized as an orthopedic compression support for a variety of injury-prone areas of the human body. Referring now to FIG. 4, the MACCSS 10 is utilized below the patella. More particularly, in this embodiment, the strap member 100 is wrapped immediately below the knee, the tip end 122 is fed through the opening 104, the distal end 108 is then fed through opening 124 and cinched with about a 2:1 mechanical advantage to the desired level of tightness and compression. The plurality of micro hoops detachably secure to the plurality of micro loops to maintain the closure compression support in the cinched position. It will be obvious to those skilled in the art that a user suffering from tennis elbow can utilize the MACCSS in the same manner as described with respect to the knee, but wrapping the strap member immediately below the elbow joint.

The orthopedic device having a mechanical advantage closure illustrated in FIG. 1, also can be used with an orthopedic support sleeve to obtain about a 2:1 mechanical advantage in the closure compression of the sleeve. Preferably, the sleeve component is composed of an elastic fabric material, e.g. neoprene. Neoprene, also known as polychloroprene, was developed in 1930 and is one of the first synthetic rubber products to be manufactured. Neoprene generally is stronger than natural rubber and more resistant to water, oils, and solvents. Of course, other materials (e.g. Lycra™ and Spandex™) may be used to manufacture the orthopedic support sleeve as will be obvious to those skilled in the art. Spandex™, Lycra™ or elastane is a synthetic fiber known for its exceptional elasticity. It is a polyether-polyurea copolymer that was invented in 1958 by chemist Joseph Shivers at DuPont.

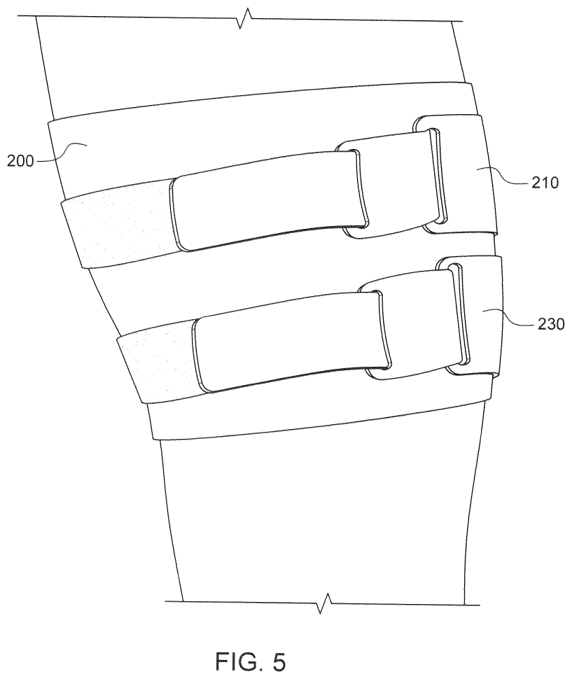

Referring now to FIG. 5, an orthopedic sleeve is shown utilizing the mechanical advantage closure compression support system of the present invention for enhancing support of the quadriceps femoris. In this embodiment, an orthopedic sleeve 200 comprises a first primary strap device 210 and a second primary strap device 230, each being sewn and stitched into the orthopedic sleeve. In operation, the user slips the sleeve 200 over the quadriceps femoris, then cinches the first primary strap device and the second primary strap device to the desired level of compression, and secures the sleeve with the hook and loop fastening means.

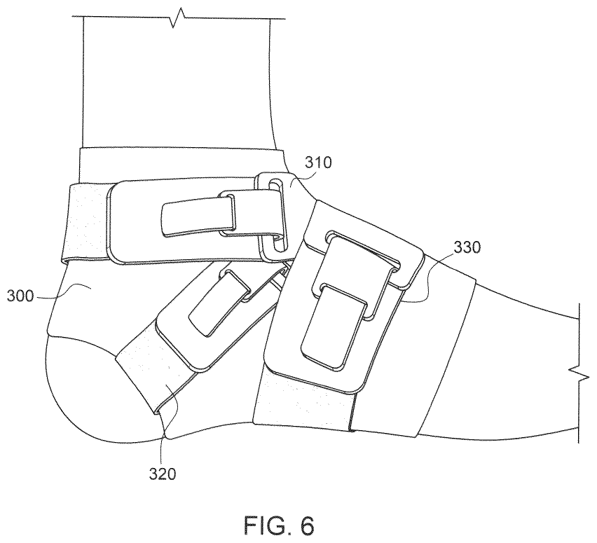

Referring now to FIG. 6, an orthopedic sleeve conforming to an ankle is shown utilizing the mechanical advantage closure compression support system of the present subject matter. In this embodiment, the ankle sleeve 300 comprises a first ankle primary strap device 310, a second hind foot strap device 320, and a third mid foot primary strap device 330. In operation, the user fits the ankle sleeve over the ankle and cinches each of the primary strap systems to the desired level of compression, securing each strap system with the hook and loop fastening system.

The foregoing detailed description of exemplary and preferred embodiments is presented for purposes of illustration and disclosure in accordance with the requirements of the law. It is not intended to be exhaustive nor to limit the invention to the precise form(s) described, but only to enable others skilled in the art to understand how the invention may be suited for a particular use or implementation. The possibility of modifications and variations will be apparent to practitioners skilled in the art. No limitation is intended by the description of exemplary embodiments which may have included tolerances, feature dimensions, specific operating conditions, engineering specifications, or the like, and which may vary between implementations or with changes to the state of the art, and no limitation should be implied therefrom. Applicant has made this disclosure with respect to the current state of the art, but also contemplates advancements and that adaptations in the future may take into consideration of those advancements, namely in accordance with the then current state of the art. It is intended that the scope of the invention be defined by the Claims as written and equivalents as applicable. Reference to a claim element in the singular is not intended to mean “one and only one” unless explicitly so stated. Moreover, no element, component, nor method or process step in this disclosure is intended to be dedicated to the public regardless of whether the element, under the provisions of 35 U.S.C § 112, sixth paragraph, unless the element is expressly recited using the phrase “means for . . . ” and no method or process step herein is to be construed under those provisions unless the step, or steps, are expressly recited using the phrase “step(s) for . . . ”

While several illustrative embodiments of the invention have been shown and described, numerous variations and alternative embodiments will occur to those skilled in the art. Such variations and alternative embodiments are contemplated and can be made without departing from the scope of the invention as defined in the appended claims.

1. An orthopedic device with a mechanical advantage closure in the form of a primary strap device comprising a strap member having an elongated length with an upper side and an underside, a proximal end having a first slotted opening, a mid-section having a plurality of micro loop fasteners on said upper side, a distal end having a plurality of micro hook fasteners on said upper side, and an arm member attached to said underside of said strap member at said mid-section and extending outwardly therefrom, said arm member having a tip end distal from said underside, said tip end having a second slotted opening.

2. The orthopedic device with a mechanical advantage closure in accordance with claim 1, wherein each of said first slotted opening and said second slotted opening is reinforced with a rectangular D-ring element.

3. The orthopedic device with a mechanical advantage closure in accordance with claim 2, wherein said rectangular D-ring element is a composed of metal.

4. The orthopedic device with a mechanical advantage closure in accordance with claim 2, wherein said rectangular D-ring element is composed of a durable plastic.

5. The orthopedic device with a mechanical advantage closure in accordance with claim 1, wherein each of said first slotted opening and said second slotted opening is in the form of a reinforced, rigid fabric material having a rectangular opening.

6. The orthopedic device with a mechanical advantage closure in accordance with claim 2, wherein said tip end is sheathed in a plastic material.

7. The orthopedic device with a mechanical advantage closure in accordance with claim 1, wherein said plurality of micro loop fasteners can be detachably secured to said plurality of micro hook fasteners.

8. The orthopedic device with a mechanical advantage closure in accordance with claim 1, wherein said arm member is attached to said strap member by stitching.

9. The orthopedic device with a mechanical advantage closure in accordance with claim 1, wherein said strap member and said arm member are composed of a material that is characterized by minimal stretch, tear resistance, lightweight, and extreme durability.

10. The orthopedic device with a mechanical advantage closure in accordance with claim 7, wherein said material is a ripstop fabric.

11. The orthopedic device with a mechanical advantage closure in accordance with claim 1, wherein an approximate 2:1 mechanical advantage is attained when said the tip end of said arm member is introduced into and fed through said first slotted opening, said distal end of said strap member is introduced into said second slotted opening and is continually pulled through said second opening such that said strap member folds back onto itself in such a manner that the plurality of micro hoops fasteners are detachably secured to said plurality of micro loops fasteners.

12. The orthopedic device with a mechanical advantage closure in accordance with claim 12, wherein said primary strap device can be utilized on injury-prone areas of the human body to provide orthopedic compression support.

13. The orthopedic device with a mechanical advantage closure in accordance with claim 12, wherein said primary strap device is utilized to provide orthopedic compression support by wrapping said strap member immediately below the knee of a user, continually pulling through said second opening, cinching to a desired level of tightness and compression, and detachably securing said plurality of micro hoop fasteners to said plurality of micro loop fasteners.

14. The orthopedic device with a mechanical advantage closure in accordance with claim 12, wherein at least one of said primary support device can be attached to an orthopedic sleeve to attain said mechanical advantage closure of said orthopedic sleeve.

15. The orthopedic device with a mechanical advantage closure in accordance with claim 14, wherein two of said primary support devices are attached to said orthopedic sleeve for enhancing compression support of the quadriceps femoris.

16. The orthopedic device with a mechanical advantage closure in accordance with claim 14, wherein three of said primary support devise are attached to said orthopedic sleeve for enhancing compression support about an ankle.

17. The orthopedic device with a mechanical advantage closure in accordance with claim 16, wherein said orthopedic sleeve is composed of an elastic fabric material.

18. The orthopedic device with a mechanical advantage closure in accordance with claim 17, wherein said elastic fabric material is neoprene.

19. The orthopedic device with a mechanical advantage closure in accordance with claim 17, wherein said elastic fabric material is selected from the group consisting of Lycra™ and Spandex™.
